(19) United States
(12) Patent Application Publication
(10) Pub. No.: US 2008/0146880 A1
(43) Pub. Date: Jun. 19, 2008

(54) Dilating Laryngoscope

(75) Inventor: Sabri E. Malek, El Paso, TX (US)

Correspondence Address: MCCARTER & ENGLISH, LLP, FOUR GATEWAY CENTER, 100 MULBERRY STREET, NEWARK, NJ, 07102, US

(73) Assignee: University of Medicine and Dentistry of New Jersey

(21) Appl. No.: 11/854,285

(22) Filed: Sep. 12, 2007

Related U.S. Application Data
(60) Provisional application No. 60/843,960, filed on Sep. 12, 2006.

(51) Int. Cl. A61B 1/267; A61M 29/02

(52) U.S. Cl. 600/194; 606/196

(57) ABSTRACT

A dilating laryngoscope which allows for dilation of a human airway (e.g., the laryngopharynx) is provided. The dilating laryngoscope includes a speculum having a pair of handles interconnected with a pair of arms and joined at a hinge. A pair of flanges are provided at the ends of the arms. An annular balloon is provided about the pair of arms, proximal to the pair of flanges. The annular balloon is connected to an air source, such as a handheld inflator connected the annular balloon by an air tube, so that the annular balloon can be inflated as desired to dilate an airway. The speculum can be manipulated by moving the handles, so that the balloon can be positioned at a desired location in the airway, and removed therefrom.

### RELATED APPLICATIONS

The present application claims the benefit of U.S. Provisional Application Ser. No. 60/843,960 filed Sep. 12, 2006, the entire disclosure of which is expressly incorporated herein by reference in its entirety.

### BACKGROUND OF THE INVENTION

1. Field of the Invention

The present invention relates to laryngoscopes, and more specifically, to a dilating laryngoscope for dilating a human airway.

2. Related Art

For endoscopists, clinicians, and other medical personnel, it is often critical to have unobstructed access to a patient's airway, such as the laryngopharynx and its neighboring structures. However, access to a patient's airway is not always easy to achieve. For example, it can often be difficult for an endoscopist to insert a fiberoptic scope (or other equipment) into a patient's airway, due to obstructions or other anatomical features which may prevent access to the airway. Similarly, examination of the human airway by an otolaryngologist or other medical personnel may also be adversely affected by obstructions or anatomical features. Perhaps most importantly, these problems can adversely affect emergency personnel during critical procedures involving the human airway, such as during intubation of a patient.

Devices for dilating (widening) orifices in the human body to allow for better access to desired anatomical structures are known in the art. One example is the speculum, which dilates a human orifice and is often employed by obstetricians and gynecologists to facilitate examination of the female anatomy. Dilation of the human airway would greatly assist medical personnel in solving the aforementioned difficulties, but existing dilation devices are inadequate for this purpose. As such, there is a need for a device which allows quick and safe dilation of a human airway to facilitate insertion of objects therein (e.g., fiberoptic scopes, endotracheal tubes, etc.) and to allow for examination of the laryngopharynx and it surrounding structures. Further, there is a need for a device which provides almost perfect, circular- or tube-like dilation of the entire circumference of a human airway, not just the sides or superior portions of the airway.

Accordingly, what would be desirable, but has not yet been provided, is a dilating laryngoscope solves the foregoing difficulties associated with accessing human airways.

### SUMMARY OF THE INVENTION

The present invention relates to a dilating laryngoscope which allows for dilation of a human airway (e.g., the laryngopharynx) to facilitate insertion of objects into the airway (e.g., fiberoptic scopes, endotracheal tubes, etc.) and to provide unobstructed access to the airway for medical examinations, etc. In one embodiment, the dilating laryngoscope includes a speculum having a pair of handles that cross each other and are joined at a pivot point. A pair of arms extend from the handles, and a pair of flanges are provided at the ends of the arms. An annular balloon is provided about the pair of arms, proximal to the flanges. The annular balloon is connected to an air source, such as a handheld inflator connected the annular balloon by an air tube, so that the annular balloon can be inflated as desired to dilate an airway. When inflated, the annular balloon provides circular or tube-like dilation of the entire circumference of the airway. The speculum can be manipulated by moving the handles, so that the balloon can be positioned at a desired location in the airway, and removed therefrom. In another embodiment, the speculum could be provided in the shape of a conventional vaginal speculum.

### BRIEF DESCRIPTION OF THE DRAWINGS

These and other important objects and features of the invention will be apparent from the following Detailed Description of the Invention, taken in connection with the accompanying drawings, in which:

FIG. 1 is a perspective view of a dilating laryngoscope constructed in accordance with the present invention.

FIG. 2 is a cross-sectional view of an annular balloon of the dilating laryngoscope of the present invention, taken along the line 2-2 of FIG. 1.

FIG. 3 is a cross-sectional view of the annular balloon of the dilating laryngoscope of the present invention, shown in an inflated state.

FIG. 4 is a partial cross-sectional view showing the dilating laryngoscope of the present invention positioned in a human airway.

FIG. 5 is a perspective view of another embodiment of the dilating laryngoscope of the present invention.

### DETAILED DESCRIPTION OF THE INVENTION

The present invention relates to a dilating laryngoscope which allows for dilation of a human airway (e.g., the laryngopharynx) to facilitate insertion of objects into the airway (e.g., fiberoptic scopes, endotracheal tubes, etc.) and to provide unobstructed access to the airway for medical examinations, etc. The dilating laryngoscope includes a speculum having a pair of handles which cross each other and which are joined at a hinge. A pair of arms extend from the handles, and a pair of flanges are provided at the ends of the arms. An annular balloon is provided about the arms, proximal to the flanges. The annular balloon is connected to an air source, such as a handheld inflator connected the annular balloon by an air tube, so that the annular balloon can be inflated as desired to dilate an airway. The speculum can be manipulated by moving the handles, so that the balloon can be positioned at a desired location in the airway, and removed therefrom.

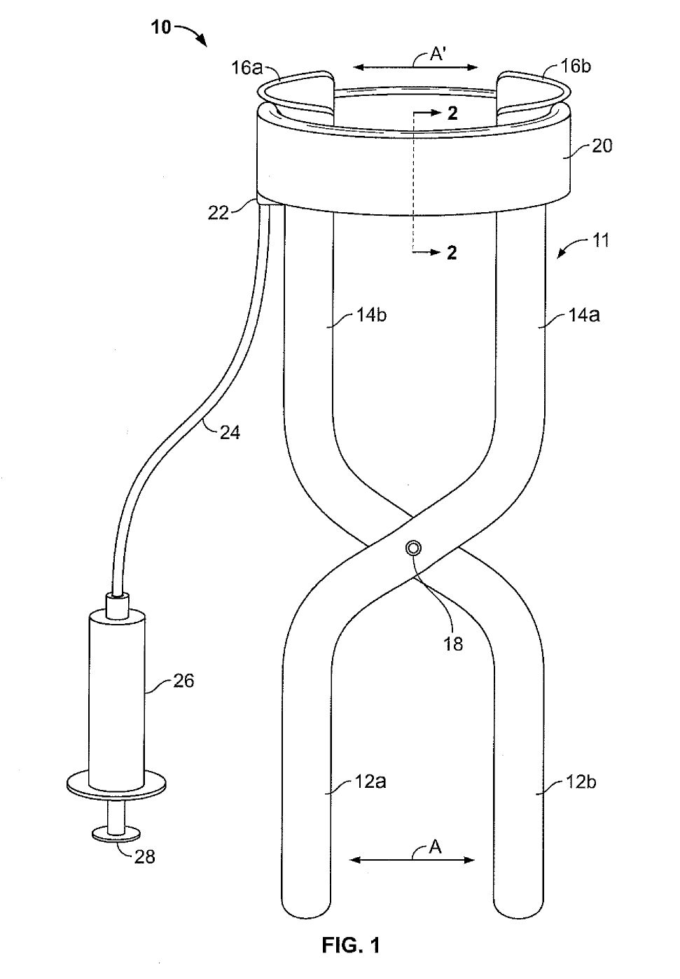

FIG. 1 is a perspective view of the dilating laryngoscope of the present invention, is indicated generally at 10. The dilating laryngoscope 10 includes a speculum 11 having a pair of handles 12a-12b which are interconnected with a pair of arms 14a-14b, respectively. A hinge 18 allows the handles 12a-12b to pivot with respect to each other (along the general direction indicated by arrow A) and to allow the arms 14a-14b to pivot with respect to each other (along the general direction indicated by arrow A′). Thus, when the handles 12a-12b are moved toward each other (along arrow A), the arms 14a-14b also move toward each other (along arrow A′). Similarly, when the handles 12a-12b are moved away from each other (along arrow A), the arms 14a-14b move away from each other (along arrow A′). The hinge 18 could comprise a pin, a screw, or any other suitable, pivotable interconnection.

The arms 14a-14b are generally flat and include widened, smooth edges and flanged ends 16a-16b, respectively. An annular balloon 20 is positioned about the arms 14a-14b, proximal to the flanged ends 16a-16b. When the dilating laryngoscope 10 is positioned in a human airway, the annular balloon 20 can be selectively inflated to allow for an unobstructed view of the larynx, trachea, and surrounding structures. The annular balloon 20 includes an air inlet port 22 connected to an air tube 24. The air tube 24 is connectable to any suitable source of air, such as a handheld inflator 26 having a plunger 28. The annular balloon 20 can thus be selectively inflated using the inflator 26. The annular balloon 20 could also be adapted for inflation using fluids, such as water or lidocane. It should also be noted that the speculum 11 could be used by itself (i.e., without the annular balloon 20) to dilate a patient's airway, due to the shape of flanged ends 16a-16b of the speculum 11 which assist with dilation of the airway when the speculum 11 is operated by a user.

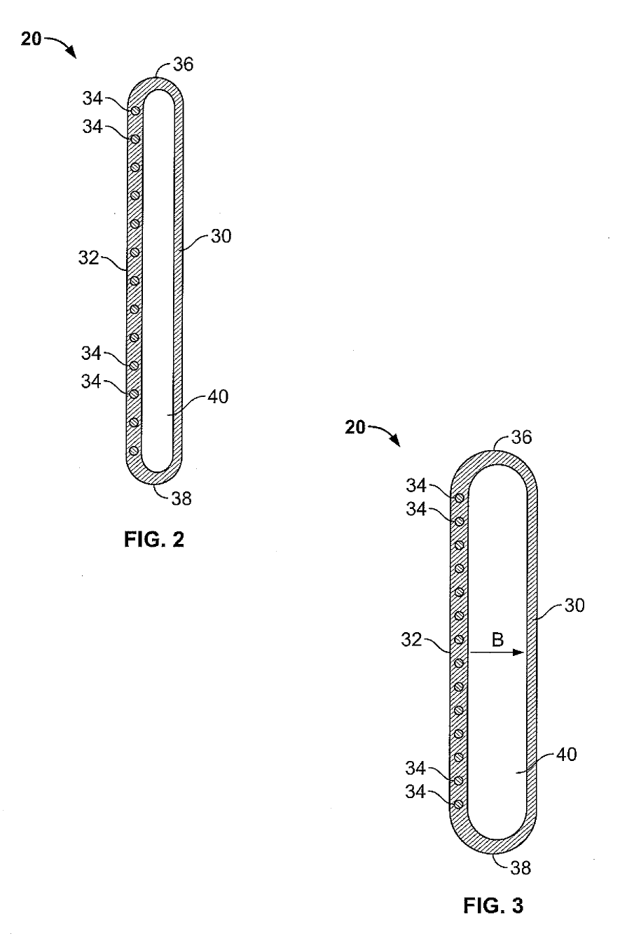

FIG. 2 is a cross-sectional view of the annular balloon 20, taken along the line 2-2 of FIG. 1. The annular balloon 20 includes an outer wall 30 and an inner wall 32 interconnected at top and bottom edges 36 and 38, respectively, to define an inflatable annular chamber 40. The inner wall 32 includes reinforcement wires 34 which provide structural support for the inner wall 32 to maintain a constant inner diameter of the annular balloon 20, particularly when the annular balloon 20 is inflated. The annular balloon 20 is approximately 2-3 millimeters thick when deflated, and approximately 3 centimeters in height. Of course, any desired dimensions or shapes could be provided without departing from the spirit or scope of the present invention. The annular balloon 20 could be manufactured from any suitable, semi-rigid plastic or other synthetic material. The reinforcement wires 34 could be manufactured from a circular or coil-like, collapsible material, such that the wires 34 could be compressed in the event that a patient swallows. Further, such material allows the wires 34 to expand to accommodate varying degrees of inflation of the annular balloon 20.

FIG. 3 is a cross-sectional view of the annular balloon 20, shown in an inflated state. As mentioned above, the reinforcement wires 34 maintain a constant inner diameter of the annular balloon 20. Thus, when the annular balloon 20 is inflated, the outer wall 30 moves away from the inner wall 32, as illustrated by arrow B. The constant inner diameter of the annular balloon 20 provides an unobstructed view of the larynx, trachea, and surrounding structures, thereby facilitating convenient access to and inspection of same, as well as insertion of medical equipment (e.g., fiberoptic scopes, endotracheal tubes, etc.) into the airway. When the annular balloon 20 is inflated, the outer wall 30 is compressed against the lining of a patient's airway, so as to retain the annular balloon 20 at a desired position in the airway and dilating the airway.

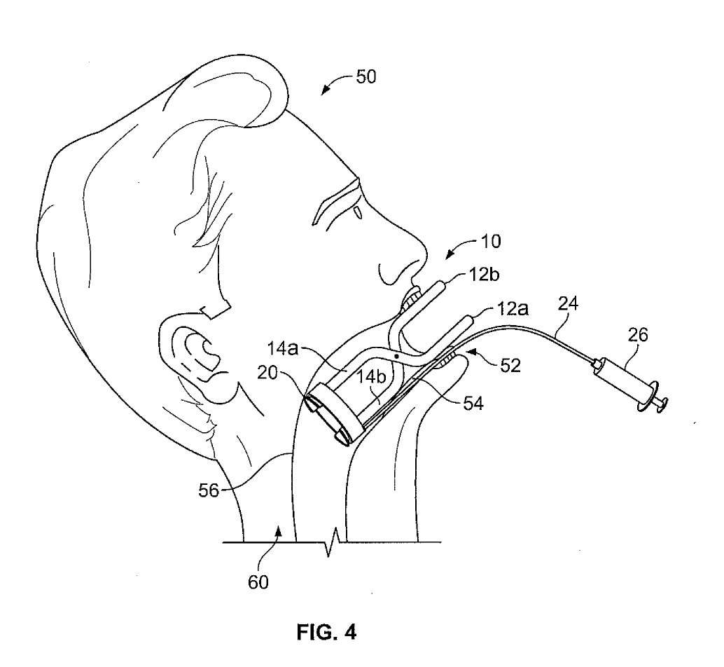

FIG. 4 is a partial cross-sectional view showing the dilating laryngoscope 10 of the present invention positioned for use in an airway 60 of a human 50. The arms 14a-14b and the annular balloon 20 are inserted past the mouth 52 and tongue 54 of the human 50. By manipulating the handles 12a-12b, the annular balloon 20 can be positioned at a desired location in the airway 60, e.g., at or near the larynx 56. The air tube 24 extends out of the mouth 52, and is connected to the inflator 26. When the annular balloon 20 is positioned at a desired location, the inflator 26 can be operated to inflate the annular balloon 20. This causes the annular balloon 20 to expand against the walls of the airway 60, thereby retaining the annular balloon 20 in position against the airway 60 and dilating the airway 60 to provide an unobstructed view of (and access to) the larynx 56 and surrounding structures. Advantageously, this greatly assists with intubation of the human 50, as well as insertion of other instruments into the airway 60, such as fiber optic devices.

After inflation of the annular balloon 20, the speculum 11 can be removed from the patients airway. In such circumstances, the annular balloon 20 would keep the airway dilated and unobstructed. If the patient were under general anesthesia, the inflated annular balloon 20 would allow the patient to breath. After a medical procedure or inspection of the airway by medical personnel, the speculum 11 could be positioned within the annular balloon 20, the annular balloon 20 could be deflated, and the speculum 11 could be used to remove the annular balloon 20 from the patient. It should be noted that the annular balloon 20 could also be permanently attached to the speculum 11.

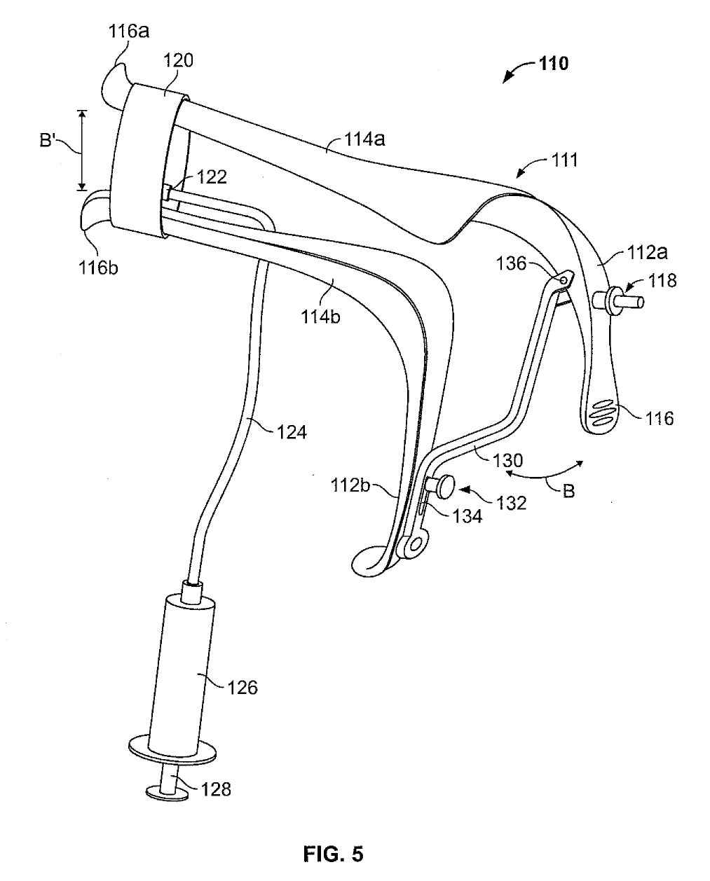

FIG. 5 is a perspective view of another embodiment of the dilating laryngoscope of the present invention, indicated generally at 110. In this embodiment, the laryngoscope 110 includes a speculum 111 in the shape of a conventional, vaginal speculum having a set of handles 112a-112b connected to a set of arms 114a-114b. The arms 114a-114b include flanged ends 116a-116b. An annular balloon 120 is positioned about the arms 114a-114b, proximal to the flanged ends 116a-116b. The balloon 120 is identical in construction to the balloon 20 described above in connection with FIGS. 1-4, and is connected to an air source, such as a handheld inflator 126 having a plunger 128 and an air tube 124 interconnecting the inflator 126 to the balloon 120. A linkage 130 interconnects the handles 112a and 112b. One end of the linkage 130 is connected to the handle 112a via a pin 136, such that the handle 112a and arm 114a can pivot with respect to the linkage 130, and the opposite end of the linkage 130 is connected to the handle 112b via an adjustable knob 132 extending through a slot 134 formed in the linkage 130. The handle 112a includes an area 116 for receiving an operator's thumb, such that when the handle 112b is grasped by an operator, the handle 112a can be moved toward the handle 112b (in the general direction indicated by arrow B). This causes the arms 114a-114b to move away from each other, in the general direction indicated by arrow B′. A knob 118 is provided on the handle 112a for adjusting the distance between the arms 114a-114b. The knob 132 also allows for adjusting the distance between the arms 114a-114b. It should be noted that the speculum 111 could be used by itself (i.e., without the annular balloon 120) to dilate a patient's airway, due to the shape of flanged ends 116a-116b of the speculum 111 which assist with dilation of the airway when the speculum 111 is operated by a user.

It should be noted that the dilating laryngoscope of the present invention could be adapted for use in a number of medical applications. Examples include, but are not limited to, vaginal examinations; rectal examinations; ear, nose, and throat examinations, procedures, and surgeries; bronchoscopy; laryngoscopy; examination of the colon; and examination of the small intestines. A smaller version of the laryngoscope of the present invention could also be provided, to allow for ear and nose examinations. Morever, the dilating laryngoscope of the present invention facilitates probe insertion for cardiologic examinations using trans-esophageal echoes, as well as placement of oral, gastric, and nasogastric tubes. Further, it should be noted that any devices suitably designed to provide for placement of an annular balloon in an orifice of the body are also within the scope of the present invention. Moreover, the present invention is not limited to applications involving the dilation of human airways. For example, the present invention could be used in veterinary applications, such as to dilate the airway of an animal. Further, the present invention could be used to dilate the airway of a human or animal cadaver (e.g., in medical education, to demonstrate proper intubation practices).

Having thus described the invention in detail, it is to be understood that the foregoing description is not intended to limit the spirit and scope thereof. What is desired to be protected by Letters Patent is set forth in the appended claims.

What is claimed is:

1. A dilating laryngoscope, comprising:
a speculum having a pair of handles interconnected with a pair of arms, and a hinge pivotally interconnecting the pair of handles;
an annular balloon positioned about the pair of arms; and
an inflator connected to the annular balloon for selectively inflating the balloon to dilate an airway.

2. A dilating laryngoscope, comprising:
a speculum having a pair of handles interconnected with a pair of arms, and an adjustable linkage pivotally interconnecting the pair of handles;
an annular balloon positioned about the pair of arms; and
an inflator connected to the annular balloon for selectively inflating the balloon to dilate an airway.

3. An annular balloon for dilating an airway, comprising:
an outer wall for contacting an airway;
an inner wall interconnected at top and bottom edges to the outer wall, the inner and outer walls defining an inflatable annular chamber;
a plurality of reinforcement wires positioned within the inner wall for maintaining a constant inner diameter of the balloon when the annular balloon is inflated; and
an air inlet port for connection to an inflator for inflating the balloon,
wherein the annular balloon dilates an airway when the annular balloon is inflated.

4. A method for dilating an airway, comprising the steps of:
providing a dilating laryngoscope including a speculum having a pair of arms, an annular balloon positioned about the pair of handles, and an inflator for selectively inflating the annular balloon;
positioning the annular balloon of the dilating laryngoscope in an airway using the speculum;
actuating the inflator to inflate the annular balloon to dilate the airway; and
removing the speculum from the airway.
